(19) United States
(12) Patent Application Publication
(10) Pub. No.: US 2026/0321430 A1
(43) Pub. Date: Oct. 15, 2026

(54) PREDICTION DEVICE, PREDICTION METHOD, AND RECORDING MEDIUM

(71) Applicant: NEC Corporation, Tokyo (JP)

(72) Inventors: Chenhui HUANG, Tokyo (JP); Kensuke WAGATA, Tokyo (JP); Fumiyuki NIHEY, Tokyo (JP)

(73) Assignee: NEC Corporation, Tokyo (JP)

(21) Appl. No.: 19/459,088

(22) Filed: Jan. 26, 2026

(30) Foreign Application Priority Data
Feb. 14, 2025 (JP) 2025-022132

(51) Int. Cl. G16H 50/30; G06N 3/045; G06N 3/08

(52) U.S. Cl. CPC G16H 50/30; G06N 3/045; G06N 3/08

(57) ABSTRACT

different modalities for one subject. The conversion unit converts data of each modality into probability distribution data indicating a probability distribution in a latent space. When data of at least one modality among the data of the plurality of modalities is missing, the imputation unit generates probability distribution data of the missing modality using a trained imputation model. The integration unit integrates the pieces of probability distribution data of the respective modalities and generates integrated probability distribution data. The prediction unit outputs a prediction result based on the integrated probability distribution data. Use of a risk prediction device of the present disclosure for predicting a disease risk can support decision-making regarding a lifestyle of a subject.

### INCORPORATION BY REFERENCE

This application is based upon and claims the benefit of priority from Japanese Patent Application 2025-022132, filed on Feb. 14, 2025, the disclosure of which is incorporated herein in its entirety by reference.

### TECHNICAL FIELD

The present disclosure relates to imputation of missing data.

### BACKGROUND ART

A prediction technology for a disease risk using a machine learning model has been known. For example, WO 2023/276976A describes a multi-modal machine learning model that predicts the progression of dementia using a plurality of types of input data.

### SUMMARY

In the multi-modal machine learning model, there is a problem in that prediction is hindered when input data of one or some modalities among the plurality of modalities is missing.

One object of the present disclosure is to provide a prediction device capable of appropriately imputing missing data if input data of one or some modalities among a plurality of modalities is missing.

According to an example aspect of the present invention, there is provided a prediction device comprising:
acquisition means configured to acquire data of a plurality of different modalities for one subject;
conversion means configured to convert data of each modality into probability distribution data indicating a probability distribution in a latent space;
imputation means configured to generate probability distribution data of the modality that is missing by using a trained imputation model, in a case where data of at least one modality among the data of the plurality of modalities is missing;
integration means configured to integrate the pieces of the probability distribution data of the respective modalities and generate integrated probability distribution data; and
prediction means configured to output a prediction result based on the integrated probability distribution data.

According to another example aspect of the present invention, there is provided a prediction method executed by a computer, the method comprising:
acquiring data of a plurality of different modalities for one subject;
converting data of each modality into probability distribution data indicating a probability distribution in a latent space;
generating probability distribution data of the modality that is missing by using a trained imputation model, in a case where data of at least one modality among the data of the plurality of modalities is missing;
integrating the pieces of the probability distribution data of the respective modalities and generating integrated probability distribution data; and
outputting a prediction result based on the integrated probability distribution data.

According to still another example aspect of the present invention, there is provided a program causing a computer to execute processing comprising:
acquiring data of a plurality of different modalities for one subject;
converting data of each modality into probability distribution data indicating a probability distribution in a latent space;
generating probability distribution data of the modality that is missing by using a trained imputation model, in a case where data of at least one modality among the data of the plurality of modalities is missing;
integrating the pieces of the probability distribution data of the respective modalities and generating integrated probability distribution data; and
outputting a prediction result based on the integrated probability distribution data.

According to the present disclosure, it is possible to appropriately impute the missing data if input data of the one or some modalities among the plurality of modalities is missing.

### BRIEF DESCRIPTION OF THE DRAWINGS

FIG. 1 illustrates an overall configuration of a risk prediction device according to the present disclosure;

FIG. 2 is a block diagram illustrating a hardware configuration of the risk prediction device;

FIG. 3 is a block diagram illustrating a functional configuration of a training device of a risk prediction model;

FIG. 4 is a flowchart of training processing of the risk prediction model;

FIG. 5 schematically illustrates a training method of an imputation model;

FIG. 6 is a flowchart of the training processing of the imputation model;

FIG. 7 is a block diagram illustrating a functional configuration of the risk prediction device;

FIG. 8 is a flowchart of risk prediction processing;

FIG. 9 is a block diagram illustrating a functional configuration of the prediction device according to the present disclosure; and

FIG. 10 is a flowchart of prediction processing according to the present disclosure.

### EXAMPLE EMBODIMENTS

Preferred example embodiments of the present disclosure will be described with reference to the accompanying drawings.

### First Example Embodiment

### [Overall Configuration]

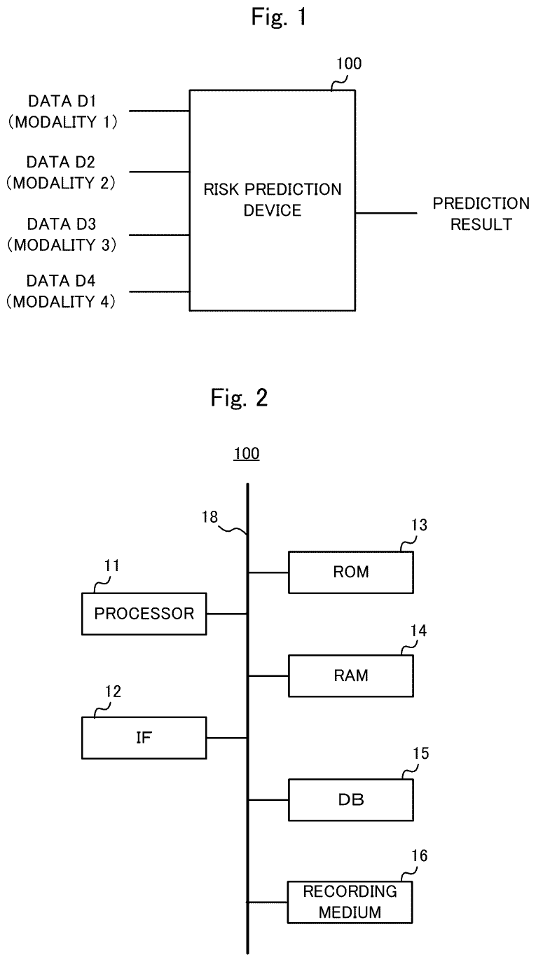

FIG. 1 illustrates an overall configuration of a risk prediction device according to the present disclosure. A risk prediction device 100 is an example of a prediction device of the present disclosure, and predicts a disease risk of a subject based on data regarding health of the subject. Specifically, multimodal data, that is, data of a plurality of different modalities is input to the risk prediction device 100. The modality means a technique, means, or the like for representing information. The multimodal data refers to data in different data formats, such as text, an image, audio, and sensor data. In the present example embodiment, the multimodal data includes, for example, various types of data obtained in a medical checkup or the like, such as the height, weight, sex, blood pressure, body mass index (BMI), body fat percentage, triglyceride level, cholesterol level, smoking status and amount, drinking status and amount, and the like of the subject.

As illustrated in FIG. 1, a plurality of pieces of data (in this example, data D1 to D4) of different modalities are input to the risk prediction device 100. The risk prediction device 100 converts the input data of the modalities into probability distributions in a latent space, and generates a probability distribution (also referred to as an “integrated probability distribution”, a “latent representation z”, or the like) obtained by aggregating the probability distributions of the respective modalities. The risk prediction device 100 then predicts a disease risk based on the integrated probability distribution and outputs a predicted result.

At the time of training, the risk prediction device 100 performs learning in such a way as to reduce an error between a predicted value of the disease risk obtained based on the integrated probability distribution and a true value of the disease risk, prepared as training data in advance. Simultaneously, the risk prediction device 100 performs training in such a way that the integrated probability distribution approaches a predetermined reference distribution (for example, a normal distribution).

At the time of the training, a machine learning model (hereinafter, also referred to as an “imputation model”) that imputes the modality data is also trained. When data of one or some modalities among the plurality of modalities is missing, it is necessary to complement the modalities that are missing (hereinafter, also referred to as “missing modalities”) in order to predict the disease risk. To this end, data of modalities other than the missing modalities is used as inputs, and based on the data, the imputation model that imputes the data of the missing modalities is generated beforehand.

On the other hand, at the time of risk prediction, the risk prediction device 100 predicts the disease risk of the subject based on the multimodal data regarding the health of the subject. Here, when data of one or some modalities among the plurality of modalities is missing, the risk prediction device 100 generates the data of the missing modalities using the imputation model trained beforehand. The risk prediction device 100 then predicts the disease risk of the subject using the data of the plurality of modalities including the imputed modality data. This enables the risk prediction device 100 to predict the disease risk with high accuracy if the data of the one or some modalities is missing.

The risk prediction device 100 can be suitably applied to medical or healthcare fields. For example, the risk prediction device 100 can be used when a risk of a lifestyle disease is predicted based on data obtained in a regular medical checkup.

### [Hardware Configuration]

FIG. 2 is a block diagram illustrating a hardware configuration of the risk prediction device 100. As illustrated in the figure, the risk prediction device 100 includes a processor 11, an interface (IF) 12, a read-only memory (ROM) 13, a random-access memory (RAM) 14, a database (DB) 15, and a storage medium 16. The components are connected through, for example, a bus 18.

The processor 11 is a computer, such as a central processing unit (CPU), and controls the entire risk prediction device 100 by executing programs prepared in advance. Specifically, as the processor 11, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these can be used.

The processor 11 loads, into the RAM 14, programs stored in the ROM 13 or the storage medium 16 and executes pieces of processing coded in the programs. The processor 11 functions as a part or all of the risk prediction device 100. Specifically, the processor 11 executes training processing and risk prediction processing, which will be described later.

The IF 12 performs transmission and reception of data to and from an external device. Specifically, in a training phase, the risk prediction device 100 receives, as training data, multimodal data on a plurality of persons through the IF 12. At the time of a prediction phase, that is, at the time of the risk prediction, the risk prediction device 100 receives the multimodal data of the subject through the IF 12 and outputs a prediction result of the disease risk to a display device or another external device.

The ROM 13 stores various programs executed by the processor 11. The RAM 14 is used as a working memory during the execution of various pieces of the processing by the processor 11.

The DB 15 stores various algorithms, data, machine learning models, and the like that are used when the risk prediction device 100 executes the training processing and the risk prediction processing, which will be described later.

The storage medium 16 is a non-volatile, non-transitory storage medium, such as a disk-shaped recording medium and a semiconductor memory. The storage medium 16 may be configured to be removable from the risk prediction device 100. The storage medium 16 records various programs executed by the processor 11.

In addition to the above, the risk prediction device 100 may include a display device, such as a liquid crystal display, and an input device, such as a keyboard and a mouse. These display device and input device are used by an operator of the risk prediction device 100, for example.

### [Training Phase]

Next, the training phase of the risk prediction device 100 will be described. In the present example embodiment, the risk prediction device 100 trains two machine learning models. A first model is a risk prediction model that predicts the disease risk from the multimodal data. A second model is the imputation model that imputes the data of the missing modalities.

### (Training of Risk Prediction Model)

First, the training of the risk prediction model will be described. The risk prediction device 100 predicts the disease risk employing a trained risk prediction model. In the following description, as an example, the risk prediction model predicts the disease risk from the data D1 to D4 of the four different modalities. However, the number of types of data constituting the multimodal data is not limited to this example.

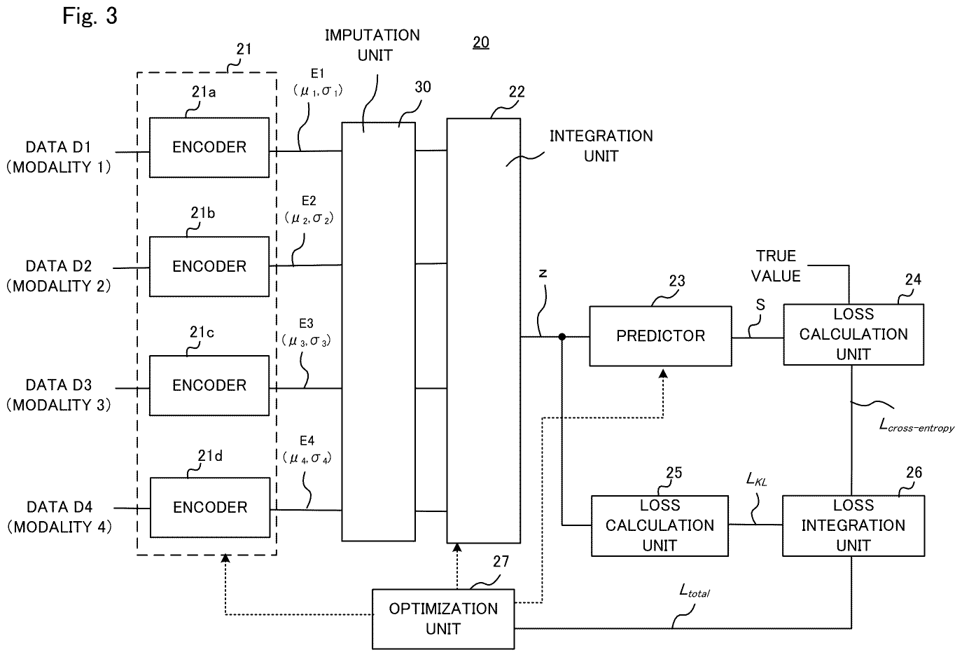

FIG. 3 is a block diagram illustrating a functional configuration of a training device 20 of the risk prediction model. The training device 20 includes an encoder unit 21, an aggregation unit 22, a predictor 23, loss calculation units 24 and 25, a loss integration unit 26, an optimization unit 27, and an imputation unit 30. The encoder unit 21 includes encoders 21a to 21d relevant to modalities 1 to 4.

The risk prediction model includes the encoder unit 21, the integration unit 22, and the predictor 23. Specifically, the encoder unit 21, the integration unit 22, and the predictor 23 are formed by neural networks. In the training phase, the training device 20 optimizes the neural networks using the training data.

As the training data, multimodal disease risk data regarding the plurality of persons is prepared. Specifically, the training data is data obtained by collecting attribute data and disease risk values of the plurality of persons. As the attribute data, for example, data, having high relevance to a disease risk to be predicted, among the height, weight, sex, blood pressure, BMI, triglyceride level, cholesterol level, blood glucose level, smoking status and amount, drinking status and amount, and the like, is used. The disease risk value of each person corresponds to ground truth data in what is called supervised learning, and is hereinafter also referred to as the “true value”. For example, it is assumed that a risk of heart disease is predicted as the disease risk with use of the blood pressure, the BMI, the triglyceride level and the cholesterol level as the data D1 to D4. In this case, data of the plurality of persons, including input data of the blood pressure, the BMI, and the triglyceride level, and including the true value of presence or absence of the heart disease, is collected as the training data.

In FIG. 3, the pieces of the data D1 to D4 of the respective modalities 1 to 4 are input to the encoder unit 21. The data D1 is input to the encoder 21a, the data D2 is input to the encoder 21b, the data D3 is input to the encoder 21c, and the data D4 is input to the encoder 21d. Each of the encoders 21a to 21d projects the input data to a latent space. The “latent space” is an abstract space for representing information contained in original data in fewer dimensions, and in the latent space, essential features and patterns of data are represented in the fewer dimensions. “Projecting to the latent space” refers to converting the original data into points on the latent space, which is also referred to as “mapping to the latent space”.

Next, each of the encoders 21a to 21d calculates a probability distribution in the latent space for each piece of the input data D1 to D4 of the respective modalities, and outputs, to the imputation unit 30, probability distribution data indicating the probability distribution. Specifically, the probability distribution data includes an average μ and a standard deviation σ. The probability distribution data (the average μ and the standard deviation σ) of each modality is also referred to as an “expert”.

When data of the plurality of input modalities is missing, the imputation unit 30 generates the data of the missing modality using data of the modalities that are not missing. Hereinafter, the modality that is missing is referred to as the “missing modality”, and the modality that is not missing is referred to as a “non-missing modality”. When the data of the plurality of input modalities is not missing, the imputation unit 30 outputs the input data as it is to the integration unit 22. Since training data of the plurality of modalities without missing is input in the training phase of the risk prediction model, the imputation unit 30 outputs the input probability distribution data of each modality as it is to the integration unit 22.

The integration unit 22 integrates the pieces of probability distribution data of the respective modalities and generates the latent representation z as the integrated probability distribution. The latent representation z is expressed by the following Expression (1), and is also referred to as an intermediate representation, a hidden representation, a latent variable, or the like.

z=μ+σ ε (1)

ε~N (0,1)
(N IS STANDARD NORMAL DISTRIBUTION)

The integration unit 22 outputs the generated latent representation z to the predictor 23 and the loss calculation unit 25.

As the integration unit 22, for example, the configuration of the product of experts (PoE) layer in the following literature can be used.

### (Document)

Microbiome-based disease prediction with multimodal variational information bottlenecks, https://doi.org/10.1371/journal.pcbi.1010050

The predictor 23 calculates a score of the disease risk (hereinafter, referred to as a “risk score”) S based on the input latent representation z, and outputs the risk score S to the loss calculation unit 24.

The loss calculation unit 24 outputs, to the loss integration unit 26, a cross entropy loss L_{cross-entropy} between the risk score S and the true value relevant to the input data D1 to D4.

The loss calculation unit 25 calculates a similarity between a reference distribution and the probability distribution that is input from the integration unit 22 and is indicated by the latent representation z. When the input data D is real number data, a normal distribution is used as the reference distribution. Hence, the loss calculation unit 25 calculates, as a loss L_{KL}, a Kullback-Leibler (KL) divergence between the probability distribution of each modality and a normal distribution N(0,1) by the following Expression (2) using an average μ and a standard deviation σ indicated by the latent representation z.

L_{KL}=D_{KL}[ƒ(N(μᵢ, σᵢ)||ƒ(N(0,1)] (2)

When the input data is not real number data, the loss calculation unit 25 can use, as the reference distribution, a log-normal distribution, a Poisson distribution, a multinomial logit, an ordinal logit, or the like according to a format of the input data D.

The loss integration unit 26 calculates a weighted sum of the loss L_{KL} and the loss L_{cross-entropy} by the following Formula (3), and outputs the weighted sum to the optimization unit 27 as a total loss Lₜₒₜₐₗ.

Lₜₒₜₐₗ=L_{KL}+λ L_{cross-entropy} (3)

“λ” indicates a weight for weighted addition of a first loss and a second loss.

The optimization unit 27 optimizes the encoder unit 21, the integration unit 22, and the predictor 23 based on the total loss Lₜₒₜₐₗ. Specifically, the optimization unit 27 optimizes parameters of the neural networks constituting the encoder unit 21, the integration unit 22, and the predictor 23 in such a way as to reduce the total loss Lₜₒₜₐₗ. Here, since the total loss Lₜₒₜₐₗ is the weighted sum of the loss L_{KL} and the loss L_{cross-entropy}, the optimization unit 27 performs optimization in such a way as to reduce the KL divergence between the reference distribution and the probability distribution that is generated by the integration unit 22 and is indicated by the latent representation, that is, in such a way as to increase the similarity between the probability distribution and the reference distribution. At the same time, the optimization unit 27 performs optimization in such a way as to reduce an error between the true value and the risk score S that is output by the predictor 23.

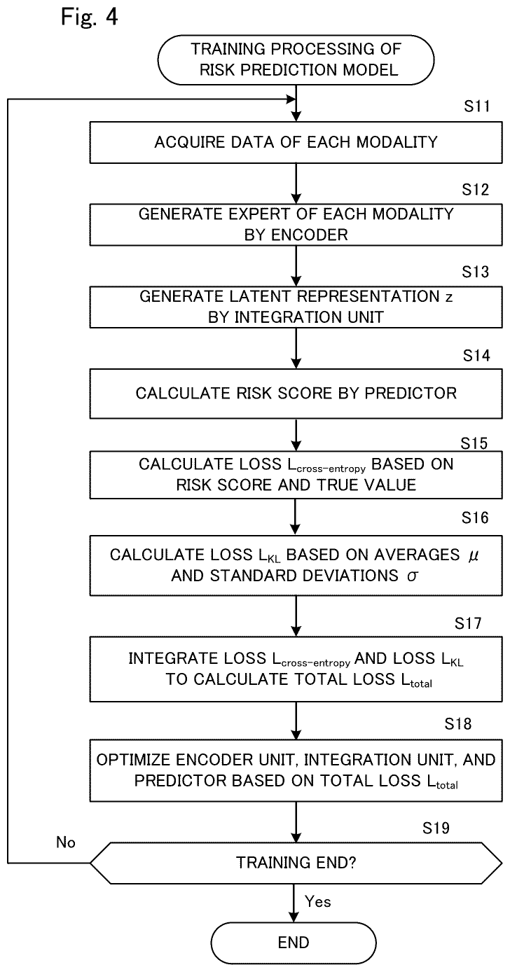

Next, the training processing executed by the above training device 20 will be described. FIG. 4 is a flowchart of the training processing of the risk prediction model. This processing is achieved by the processor 11 illustrated in FIG. 2 executing the programs prepared in advance and operating as the components illustrated in FIG. 3.

First, the encoder unit 21 acquires the data of each modality contained in the training data (step S11). Next, the encoder unit 21 projects each piece of the data to the latent space by each of the encoders 21a to 21d, and generates the expert (a pair of the average μ and the standard deviation σ) of each modality (step S12). At the time of the training, since the input data of each modality is not missing, the expert of each modality is input to the integration unit 22 via the imputation unit 30. Next, the integration unit 22 integrates the experts of the respective modalities and generates the latent representation z in the latent space (step S13). Next, the predictor 23 calculates the risk score S based on the latent representation z (step S14).

Next, the loss calculation unit 24 calculates the loss L_{cross-entropy} based on the risk score S and the true value (step S15). The loss calculation unit 25 also calculates the loss L_{KL} using the average μ and the standard deviation σ of each modality (step S16). Next, the loss integration unit 26 calculates the total loss Lₜₒₜₐₗ from the loss L_{cross-entropy} and the loss L_{KL} (step S17). Next, the optimization unit 27 optimizes the parameters of the encoder unit 21, the integration unit 22, and the predictor 23 based on the total loss Lₜₒₜₐₗ (step S18).

Next, the training device 20 determines whether a predetermined training end condition is satisfied (step S19). Examples of the training end condition include one that a predetermined number of pieces of the attribute data prepared as the training data is used, one that the total loss becomes equal to or less than a predetermined value, one that the total loss converges, or the like. When the training end condition is not satisfied (step S19: No), the processing returns to step S11.

On the other hand, when the training end condition is satisfied (step S19: Yes), the training processing ends. In this way, the risk prediction model is generated.

### (Training of Imputation Model)

Next, the training of the imputation model will be described. When data of the plurality of input modalities is missing, the imputation model generates the data of the missing modality from the data of the non-missing modalities.

In the present example embodiment, the imputation model is created using a language model. The language model is a mathematical model for generating a sentence or a part of a sentence or evaluating validity of a sentence in the field of natural language processing, and is specifically a model for predicting a word or a sentence that comes next in a given context. Hence, in the present example embodiment, the expert of each modality output by the encoder unit 21 is considered as a vectorized token. That is, the expert of each modality is input to the imputation model as the token. When one or some modalities among the plurality of input modalities are missing, the imputation model predicts tokens of the missing modalities based on tokens of the non-missing modalities, and outputs predicted tokens. This allows the imputation model to impute the missing modalities.

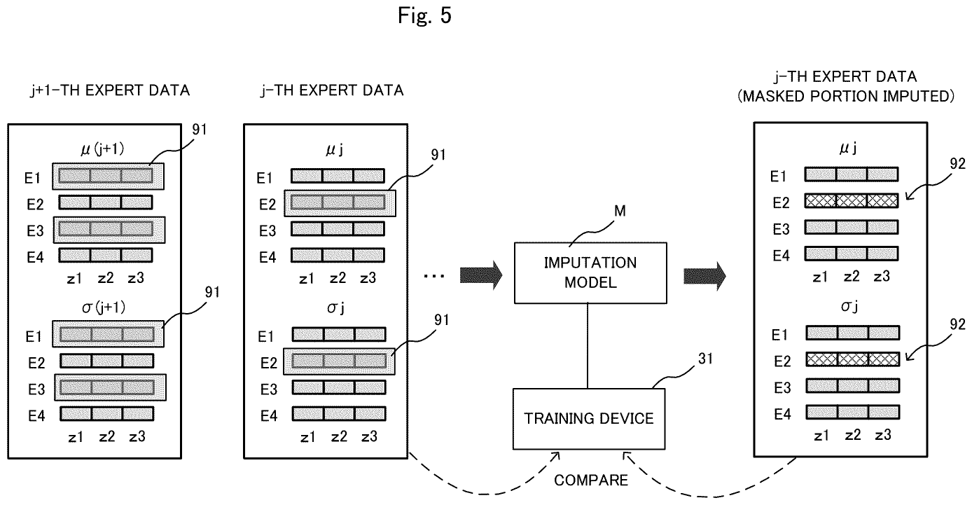

FIG. 5 schematically illustrates a training method of the imputation model. First, the experts will be described. Now, assuming that a certain subject among a plurality of subjects is indicated by “j”, j-th training data relevant to this subject contains data of the four modalities. In FIG. 3, experts (each including the pair of the average μ and the standard deviation σ) E1 to E4 relevant to the respective modalities are output from the encoders 21a to 21d. Now, it is assumed that the latent representation z in the latent space is three-dimensional. The experts for the certain subject j each include the average μj of the subject j and the standard deviation σj of the subject j. Here, as illustrated in FIG. 5, the average μj can be represented by a 4×3 matrix in which the number of dimensions z1 to z3 is taken in a row direction and the experts E1 to E4 are taken in a column direction. Similarly, the standard deviation σj of the subject j can be represented by a 4×3 matrix in which the number of dimensions z1 to z3 is taken in the row direction and the experts E1 to E4 are taken in the column direction.

Next, description will be given of the training of the imputation model. At the time of the training, a training device 31 functioning as training means inputs the j-th expert data relevant to the subject j to the imputation model. At this time, the training device 31 randomly masks one or some modalities of the input expert data. The masking here corresponds to intentionally creating a missing modality. In the example of FIG. 5, as indicated by reference signs 91, the training device 31 masks the expert E2 of the second modality in the j-th expert data, and inputs, to an imputation model M, the average μj and the standard deviation σj in each of which the expert E2 is missing.

The imputation model M predicts, from the data of the other experts, the data of the masked expert in the input. The imputation model M configured using the language model handles each input expert as the token and predicts the masked expert. In FIG. 5, the imputation model M predicts the data of the masked expert E2, as indicated by reference signs 92. Then, the training device 31 trains the imputation model M in such a way as to reduce a difference between data of the expert E2 predicted by the imputation model M and the data of the masked expert E2 as indicated by the reference signs 91. In this way, the training is performed by randomly masking the one or some of the four modalities in the plurality of pieces of expert data, thereby obtaining the imputation model that generates the data of the masked modality (that is, the missing modality).

As the language model constituting the imputation model, for example, a masked language model (hereinafter, referred to as “MLM”) of bidirectional encoder representations from transformers (BERT) can be used. However, the imputation model is not limited to the above MLM, and any language model may be used as long as a missing portion of the input tokens when one or some of the input tokens are missing can be predicted.

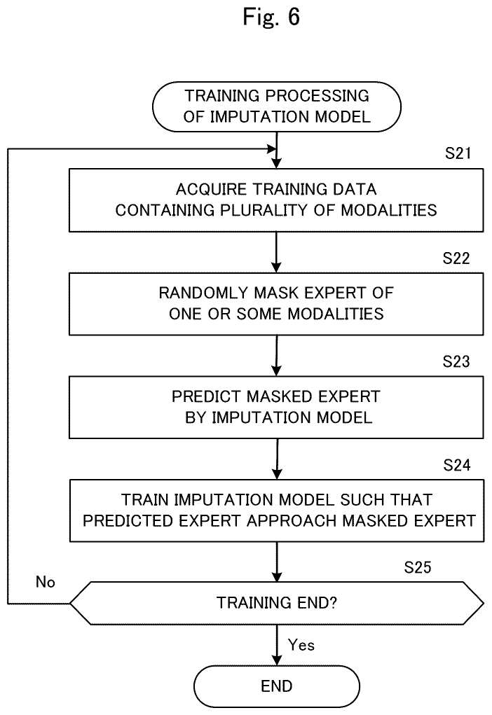

FIG. 6 is a flowchart of the training processing of the imputation model. This processing is achieved by the processor 11 illustrated in FIG. 2 executing the program prepared in advance and operating as the training device 31 illustrated in FIG. 5.

First, the training device 31 acquires the training data containing the plurality of modalities (step S21). Next, the training device 31 randomly masks the expert of the one or some modalities in the training data (step S22). Next, the training device 31 inputs the partially masked training data to the imputation model and predicts the masked expert (step S23). Next, the training device 31 trains the imputation model in such a way that the predicted expert approaches the masked expert (step S24).

Next, the training device 31 determines whether a predetermined training end condition is satisfied (step S25). Examples of the training end condition include one that a predetermined number of the pieces of data prepared as the training data is used, one that an error between the predicted expert and the masked expert is equal to or less than a predetermined value, or the like. When the training end condition is not satisfied (step S25: No), the processing returns to step S21, and the training is continued using next training data. On the other hand, when the training end condition is satisfied (step S25: Yes), the processing ends.

### [Prediction Phase]

Next, the prediction phase by the risk prediction device will be described. In the prediction phase, the risk prediction device 100 predicts the disease risk of a certain subject based on multimodal data of the subject. At this time, the risk prediction device 100 uses the risk prediction model trained in the training phase, specifically, the encoder unit 21, the integration unit 22, and the predictor 23. When one or some modalities of the multimodal data of the certain subject are missing in the prediction phase, the risk prediction device 100 predicts the risk after the data of the missing modalities is imputed using the trained imputation model.

### (Risk Prediction Device)

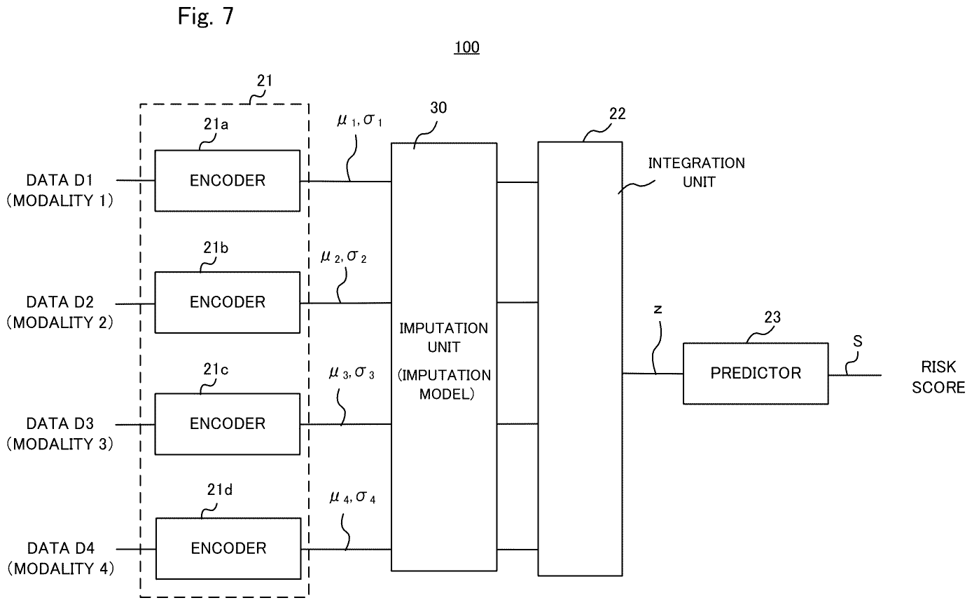

FIG. 7 is a block diagram illustrating a functional configuration of the risk prediction device. The risk prediction device 100 includes the encoder unit 21, the integration unit 22, and the predictor 23 that are optimized in the training phase. The risk prediction device 100 also includes the imputation unit 30 for imputing the data of the missing modalities. The trained imputation model is used as the imputation unit 30.

In the above configuration, the encoder unit 21 is an example of acquisition means and conversion means, the integration unit 22 is an example of integration means, the predictor 23 is an example of prediction means, and the imputation unit 30 is an example of imputation means.
(I) When modality is not missing

First, a case where the modalities in the input data are not missing will be described. In this case, for the certain subject, pieces of data D1 to D4 of the four different modalities are input to the encoder unit 21. The encoders 21a to 21d project the input data D1 to D4 to the latent space, generate probability distribution data (experts) containing averages μ and standard deviations σ, and output the probability distribution data to the imputation unit 30. In the case where the modalities are not missing, the imputation unit 30 outputs the pieces of input probability distribution data of the respective modalities to the integration unit 22 as they are.

The integration unit 22 integrates the pieces of probability distribution data of the respective modalities, and generates a latent representation z as an integrated probability distribution obtained by integrating probability distributions of the respective modalities. The integration unit 22 outputs the latent representation z to the predictor 23.

The predictor 23 calculates a risk score S indicating the disease risk based on the input latent representation z, and outputs the result. In this way, the disease risk of the subject can be predicted based on the multimodal data.
(II) When modality is missing

Next, a case where a modality in the input data is missing will be described. In this case, the imputation unit 30 generates data of the missing modality using the trained imputation model. When an expert of the missing modality is imputed by the imputation unit 30, the risk prediction device 100 predicts the disease risk of the subject using the imputed expert. Processing of the risk prediction device 100 subsequent to the imputation of the missing expert is similar to the processing in the case where the input data is not missing.

### (Risk Prediction Processing)

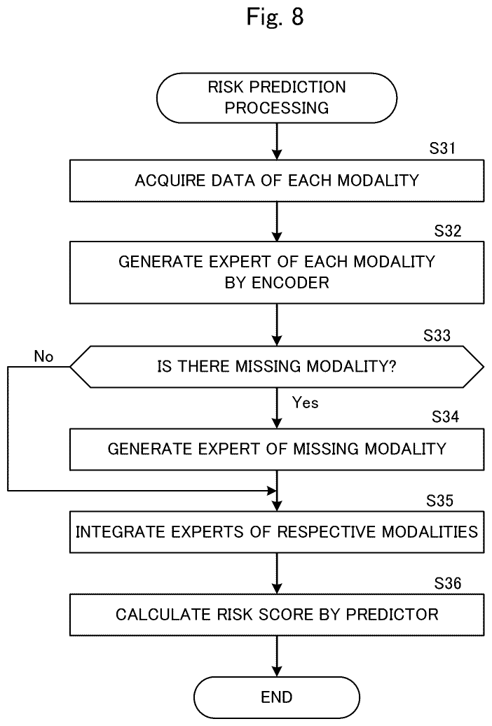

Next, the risk prediction processing executed by the above risk prediction device 100 will be described. FIG. 8 is a flowchart of the risk prediction processing. This processing is achieved by the processor 11 illustrated in FIG. 2 executing the programs prepared in advance and operating as the components illustrated in FIG. 7.

First, the risk prediction device 100 acquires the input data of each modality for the subject (step S31). Next, the encoder unit 21 generates the expert of each modality from the input data of each modality (step S32). The encoder unit 21 does not generate the expert for the missing modality here. That is, in step S32, the experts of the non-missing modalities are generated.

Next, the risk prediction device 100 determines whether there is a missing modality in the input data (step S33). When there is no missing modality (step S33: No), the processing proceeds to step S35. On the other hand, when there is a missing modality (step S33: Yes), the risk prediction device 100 generates an expert of the missing modality by the imputation unit 30 (step S34).

Next, the integration unit 22 integrates the experts of the respective modalities and generates the latent representation z in the latent space (step S35). When data of the input data is missing, the integration unit 22 integrates the experts of the non-missing modalities generated in step S32 and the expert of the missing modality generated in step S34. Next, the predictor 23 calculates the risk score S based on the latent representation z and outputs the result (step S36). Then, the risk prediction processing ends.

### [Modifications]

### (First Modification)

In the above first example embodiment, the predictor 23 predicts the risk of the disease of the subject based on the latent representation z after the integration. In this case, the predictor 23 predicts the risk of heart disease from, for example, the height, weight, blood pressure, and cholesterol level and outputs the result.

Alternatively, a predictor 23 may be configured to predict another piece of health data of a subject. Specifically, the predictor 23 is configured to predict health data of a modality different from the modalities of the input data D1 to D4. In this case, the predictor 23 may predict a triglyceride level from, for example, the height, weight, blood pressure, and cholesterol level, and output a result.

Alternatively, the predictor 23 may be configured to predict health data of a missing modality of the subject. Specifically, the predictor 23 is configured to output data of a missing modality among input data D1 to D4. In this case, for example, when the input data D3 (blood pressure) among the input data D1 (height), D2 (weight), D3 (blood pressure), and D4 (cholesterol level) is missing, the predictor 23 may predict and output the input data D3 (blood pressure).

### (Second Modification)

In the above first example embodiment, the risk prediction device is applied to the generation of the attribute data regarding human health. However, the application of the present disclosure is not limited thereto. For example, the present disclosure may be applied to inspection and diagnosis of machines and devices. That is, the technique of the present disclosure may be applied when a state of the machine or device is estimated based on data of a plurality of modalities detected and collected in the inspection or the diagnosis.

### Second Example Embodiment

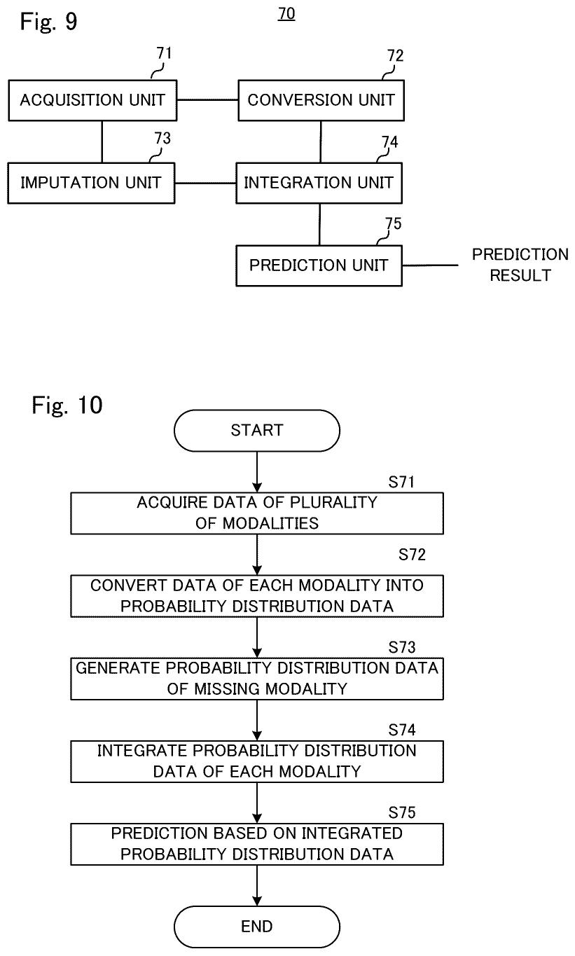

FIG. 9 is a block diagram illustrating a functional configuration of a risk prediction device of a second example embodiment. A prediction device 70 includes an acquisition unit 71, a conversion unit 72, an imputation unit 73, an integration unit 74, and a prediction unit 75.

FIG. 10 is a flowchart of processing by the prediction device of the second example embodiment. The acquisition unit 71 acquires data of a plurality of different modalities for one subject (step S71). The conversion unit 72 converts data of each modality into probability distribution data indicating a probability distribution in a latent space (step S72). When data of at least one modality among the data of the plurality of modalities is missing, the imputation unit 73 generates probability distribution data of the missing modality using a trained imputation model (step S73). The integration unit 74 integrates the pieces of probability distribution data of the respective modalities and generates integrated probability distribution data (step S74). The prediction unit 75 outputs a prediction result based on the integrated probability distribution data (step S75).

According to the prediction device 70 of the second example embodiment, it is possible to predict a risk with high accuracy if part of the input data is missing.

A part or all of the example embodiments described above may also be described as the following supplementary notes, but not limited thereto.

### (Supplementary Note 1)

A prediction device comprising:
acquisition means configured to acquire data of a plurality of different modalities for one subject;
conversion means configured to convert data of each modality into probability distribution data indicating a probability distribution in a latent space;
imputation means configured to generate probability distribution data of the modality that is missing by using a trained imputation model, in a case where data of at least one modality among the data of the plurality of modalities is missing;
integration means configured to integrate the pieces of the probability distribution data of the respective modalities and generate integrated probability distribution data; and
prediction means configured to output a prediction result based on the integrated probability distribution data.

### (Supplementary Note 2)

The prediction device according to Supplementary note 1, wherein the imputation model generates the probability distribution data of the modality that is missing, based on data of a modality that is not missing among the plurality of modalities.

### (Supplementary Note 3)

The prediction device according to Supplementary note 2, wherein the imputation model is a language model that uses probability distribution data of each modality as an input and outputs probability distribution data of a modality that is missing.

### (Supplementary Note 4)

The prediction device according to Supplementary note 1, further comprising training means for training the imputation model by machine learning with use of training data of the plurality of modalities.

### (Supplementary Note 5)

The prediction device according to Supplementary note 4, wherein the training means masks data of one or some modalities among the training data of the plurality of modalities and inputs the masked data to the imputation model, and trains the imputation model based on prediction data output by the imputation model and the masked data.

### (Supplementary Note 6)

The prediction device according to Supplementary note 1, wherein the prediction means predicts a disease risk of a subject based on data of a plurality of modalities regarding health of the subject.

### (Supplementary Note 7)

The prediction device according to Supplementary note 1, wherein the prediction means predicts data of another modality regarding health of a subject based on data of a plurality of modalities regarding health of the subject.

### (Supplementary Note 8)

The prediction device according to Supplementary note 1, wherein the prediction means predicts data of a modality that is missing, based on data of a modality that is not missing among data of a plurality of modalities regarding health of the subject.

### (Supplementary Note 9)

A prediction method executed by a computer, the method comprising:
acquiring data of a plurality of different modalities for one subject;
converting data of each modality into probability distribution data indicating a probability distribution in a latent space;
generating probability distribution data of the modality that is missing by using a trained imputation model, in a case where data of at least one modality among the data of the plurality of modalities is missing;
integrating the pieces of the probability distribution data of the respective modalities and generating integrated probability distribution data; and
outputting a prediction result based on the integrated probability distribution data.

### (Supplementary Note 10)

A program causing a computer to execute processing comprising:
acquiring data of a plurality of different modalities for one subject;
converting data of each modality into probability distribution data indicating a probability distribution in a latent space;
generating probability distribution data of the modality that is missing by using a trained imputation model, in a case where data of at least one modality among the data of the plurality of modalities is missing;
integrating the pieces of the probability distribution data of the respective modalities and generating integrated probability distribution data; and
outputting a prediction result based on the integrated probability distribution data.

Some or all of the configurations described in Supplementary Notes 2 to 8 dependent on the above-described Supplementary Note 1 can also be dependent on Supplementary Notes 9 and 10 by a dependency relationship similar to that of Supplementary Notes 2 to 8. Some or all of the configurations described as the Supplementary Notes can be similarly dependent on not only the Supplementary Notes 1, 9, and 10, but also diverse pieces of hardware and software, various recording means for recording software, or systems without departing from the above-described example embodiments.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure. In other words, the present disclosure naturally includes various modifications and alterations that a person skilled in the art would be able to make in accordance with the entire disclosure, including the scope of the claims, and the technical ideas.

### DESCRIPTION OF SYMBOLS

11 Processor
20 Training device
21 Encoder unit
21a to 21d Encoder
22 Integration unit
23 Predictor
24, 25 Loss calculation unit
26 Loss integration unit
27 Optimization unit
30 Imputation unit
31 Training device
100 Risk prediction device

1. A prediction device comprising:
an interface configured to receive data of a plurality of different modalities for one subject;
a memory configured to store instructions; and
a processor configured to execute the instructions to:
acquire, via the interface, the data of the plurality of different modalities for the subject;
map, for each modality, the data acquired via the interface to a latent space and generate probability distribution data including at least a mean and a standard deviation of a probability distribution in the latent space;
generate, in a case where data of at least one modality among the data of the plurality of modalities is missing, probability distribution data of the missing modality by using a trained imputation model;
integrate the pieces of the probability distribution data of the respective modalities by using a product-of-experts layer and generate a latent representation in the latent space; and
output a prediction result based on the latent representation.

2. The prediction device according to claim 1, wherein the imputation model generates the probability distribution data of the modality that is missing, based on data of a modality that is not missing among the plurality of modalities.

3. The prediction device according to claim 2, wherein the imputation model is a language model that uses probability distribution data of each modality as an input and outputs probability distribution data of a modality that is missing.

4. The prediction device according to claim 1, wherein the processor is further configured to execute the instructions to train the imputation model by machine learning with use of training data of the plurality of modalities.

5. The prediction device according to claim 4, wherein the processor masks data of one or some modalities among the training data of the plurality of modalities and inputs the masked data to the imputation model, and trains the imputation model based on prediction data output by the imputation model and the masked data.

6. The prediction device according to claim 1, wherein the prediction means predicts a disease risk of a subject based on data of a plurality of modalities regarding health of the subject.

7. The prediction device according to claim 1, wherein the processor predicts data of another modality regarding health of a subject based on data of a plurality of modalities regarding health of the subject.

8. The prediction device according to claim 1, wherein the processor predicts data of a modality that is missing, based on data of a modality that is not missing among data of a plurality of modalities regarding health of the subject.

9. A prediction method executed by a computer, comprising:
acquiring, via an interface, data of a plurality of different modalities for one subject;
mapping, for each modality, the data acquired via the interface to a latent space and generating probability distribution data including at least a mean and a standard deviation of a probability distribution in the latent space;
generating, in a case where data of at least one modality among the data of the plurality of modalities is missing, probability distribution data of the missing modality by using a trained imputation model;
integrating the pieces of the probability distribution data of the respective modalities by using a product-of-experts layer and generating a latent representation in the latent space; and
outputting a prediction result based on the latent representation.

10. A non-transitory computer-readable recording medium storing a program, the program causing a computer to execute processing comprising:
acquiring, via an interface, data of a plurality of different modalities for one subject;
mapping, for each modality, the data acquired via the interface to a latent space and generating probability distribution data including at least a mean and a standard deviation of a probability distribution in the latent space;
generating, in a case where data of at least one modality among the data of the plurality of modalities is missing, probability distribution data of the missing modality by using a trained imputation model;
integrating the pieces of the probability distribution data of the respective modalities by using a product-of-experts layer and generating a latent representation in the latent space; and
outputting a prediction result based on the latent representation.
